(19) United States
(12) Patent Application Publication
(10) Pub. No.: US 2025/0120681 A1
(43) Pub. Date: Apr. 17, 2025

(54) APPARATUS AND METHOD FOR THE DIAGNOSIS OF VULVOVAGINAL CONDITIONS

(71) Applicant: MOR RESEARCH APPLICATIONS LTD., Ramat Gan (IL)

(72) Inventor: Arnon AGMON, Givaataym (IL)

(21) Appl. No.: 18/686,446

(22) PCT Filed: Aug. 25, 2022
(86) PCT No.: PCT/IB2022/057958

§ 371 Date: Feb. 26, 2024

(30) Foreign Application Priority Data
Aug. 26, 2021 (SG) 10202109369P

(51) Int. Cl. A61B 10/00; G01N 33/543

(52) U.S. Cl. CPC A61B 10/0045; G01N 33/54388; A61B 2010/0074; G01N 2800/36

(57) ABSTRACT

A system, apparatus and method for diagnosis of vaginal disorders, comprising a collection apparatus for collecting and sampling vaginal discharge or fluid and a sensing device for processing the sampling vaginal discharge or fluid, said sensing device comprising at least two lateral flow assays (LFAs).

### CROSS REFERENCE TO RELATED APPLICATIONS

The present application claims the benefit of the Singapore Application No. 10202109369P filed on 26 Aug. 2021, the disclosure of which is hereby incorporated in its entirety by reference herein.

### TECHNICAL FIELD

The present disclosure relates to a medical diagnosis apparatus and method and particularly to the diagnosis of common vulvovaginal infections and vulvar contact dermatitis.

### BACKGROUND

The most common reason for Gynecological visits by adult women is due to various forms of vulvovaginal infections. The most common vaginal infections include Bacterial Vaginosis, Candida infection and Trichomonas infection. The most common reason for vulvar complaints is contact dermatitis. Vulvovaginal conditions are commonly associated with very disturbing symptoms which makes this condition an urgent matter for the sufferer. The increasing low availability of physicians and long wait time for appointment induce self-diagnosis and self-medication which in most instances not curative and lowers diagnosis accuracy by physician.

It is estimated that about half of all professional diagnosis of Vaginitis is erroneous. This high diagnosis failure rate is likely due to overlapping signs and symptoms of the various vaginal conditions. Other factors affecting diagnostic accuracy include a relatively long diagnostic procedure, around 20 minutes, a need for multiple tests and tools (pH meter, KOH test, Microscopy, culture, Immunoassays, DNA tests and so forth) as well as medical expertise which most practicing gynecologists do not possess. This high diagnostic failure rate leads to incorrect therapy, worsening of condition, increased suffering and repeat visits. The arduous diagnostic process is also in the heart of a vicious cycle of low diagnostic performance leading to therapy failure leading to repeat visits, high cost for insurer and increased patient suffering. The use of DNA tests is expensive and is found only in large Points Of Care (POC) thus not readily available to many community based clinics. Therefore, there is a need for a simple, accurate and cheap diagnostic tool for vulvovaginal infections.

Telemedicine has become very common in recent years, and especially during and following the Coronavirus (COVID-19) pandemic. Both patients and physicians accept the use of telemedicine as a part of providing and receiving health services. Many Health Maintenance Organizations (HMOs) are now providing financial incentives for physicians to engage with patients using telemedicine. This saves time and money for all parties involved. With the aim of providing more services via telemedicine, especially those requiring diagnosis of medical conditions, various accessory devices are already in the market today. These include devices such as Tyto for internal medicine diagnostic purposes, remote digital urine test for the diagnosis of Urinary Tract Infection, both used by large HMOs. In the field of vulvovaginal infections, there are various home diagnostic tests including pH meters which provide a rough estimate of vaginal pH, several Lateral Flow immune Assays (LFA) which provides fast and reliable results for the presence of individual organisms or enzymatic activity. Recently a new Helicase dependent DNA detection technique tests for individual organisms was also introduced. These devices were designed originally to be used individually by physicians, require multiple samples and multiple preparation steps. When considering the information required for the diagnosis of vaginal infections, at least three sample and three different preparation processes are required. Consequently, these tests are not cost effective for telemedicine use due to their handling and time constraints. Therefore, there is a need for a comprehensive, accurate, fast diagnostic tool for home use that can be efficiently used in telemedicine.

### SUMMARY

An aspect of an embodiment of the disclosure, relates to an apparatus and method for diagnosis of vaginal disorders, such as common vulvovaginal infections. The apparatus and method comprise of multiple inputs including physical, chemical and organism detection as well as visual images, to provide information pertinent for the diagnosis of vaginal infection.

There is therefore provided an apparatus for diagnosis of vaginal disorders, comprising a collection apparatus for collecting and sampling vaginal discharge or fluid and a sensing device for processing the sampling vaginal discharge or fluid, said sensing device comprising at least two lateral flow assays (LFAs). In an embodiment of the disclosure, the apparatus may further include a visual data acquisition device for capturing images of the vulva, sampling vaginal discharge or fluid and results generated by the at least two LFAs. The apparatus further comprising a central processing unit, a memory storage, and a communication device.

In an embodiment of the disclosure, the collection apparatus is shaped to be handheld and to comfortably fit the vagina. The collection apparatus further comprises a vaginal end with at least one swab and at least one pH sensor. In some embodiments, the collection apparatus is shaped as a spoon and comprises a handle and a collection cup having an opening. The collection apparatus further comprises a pH sensor mounted such that when a sample is collected the pH sensor is immersed in vaginal fluid. The pH sensor may be immersed partially or fully in vaginal fluid. The pH sensor is comprised of at least one wall made of transparent material.

In an embodiment of the disclosure, the at least two LFAs is selected from the group consisting of tests or kits for Candida species antigenic, Trichomonas vaginalis antigenic, sialidase enzyme activity, Chlamydia trachomatis antigenic, rapid nucleic acid detection of microorganism presence, detection of vaginal amines molecules typical of vaginitis. The sensing device further comprises a portable spectrometer.

In an embodiment of the disclosure, the visual data acquisition is a camera for capturing images of vulva, discharge, and vulvovaginal fluid.

In an embodiment of the disclosure, the sensing device comprises a housing having a top part and a flat base, the top part is elevated compared with the flat based, allowing for mixed vaginal fluids to passively flow to the at least two LFAs. The housing has a shape that comport to the shape of collection apparatus. The sensing device further comprises a sampling and mixing chamber formed of walls and a bottom at a proximal end and at least two view ports at the distal end. The sensing device further comprises a delivery mechanism, the delivery mechanism comprises a delivery conduit connecting the sampling and mixing chamber and the and at least two LFAs. The sensing device further comprises a central processing unit, a memory storage, and a communication device.

In an embodiment of the disclosure, the sampling and mixing chamber further comprises a buffer solution or reagents for the preparation of collected sample prior to delivery to the at least two LFAs. The sampling and mixing chamber further comprises a mixing mechanism. The delivery mechanism comprises at least two tubes extending from the sampling and mixing chamber.

In an embodiment of the disclosure, the sampling and mixing chamber may comprise at least two sampling and mixing chambers separated by a divider wall. The two sampling and mixing chambers may be placed on a rotatable base thereby facilitating the mixing of the solution contained in the chamber with the sample. The two sampling and mixing chambers may further comprise puncture elements, wherein apertures are formed when said puncture elements are pushed through walls of the sampling and mixing chamber.

There is further provided a kit for diagnosing vaginal disorders comprising a collection apparatus for collecting and sampling vaginal discharge or fluid and a sensing device for processing the sampling vaginal discharge or fluid, wherein said sensing device comprising at least two LFAs. In an embodiment of the disclosure, the kit comprises the at least two LFAs that is selected from the group consisting of tests or kits for Candida species antigenic, Trichomonas vaginalis antigenic, sialidase enzyme activity, Chlamydia trachomatis antigenic, rapid nucleic acid detection of microorganism presence, and detection of vaginal amines molecules typical of vaginitis.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a system for diagnosis of vaginal disorders, according to an embodiment of the disclosure;

FIG. 2 is a schematic illustration of collection apparatus in the form of a collecting spoon, according to an embodiment of the disclosure;

FIG. 3A is a perspective view of a sensing device, according to an embodiment of the disclosure;

FIG. 3B is a cross sectional view AA′ of the perspective view of the sensing device in FIG. 3A, according to an embodiment of the disclosure;

FIG. 4A is a perspective view of the sensing device in accordance with an embodiment of the present disclosure, wherein the sensing device has two sampling and mixing chambers;

FIG. 4B is a lateral view of the sensing device, according to an embodiment of the disclosure, wherein the sensing device has two sampling and mixing chambers;

FIG. 4C is a top view of the two sampling and mixing chambers, according to an embodiment of the disclosure.

### DETAILED DESCRIPTION

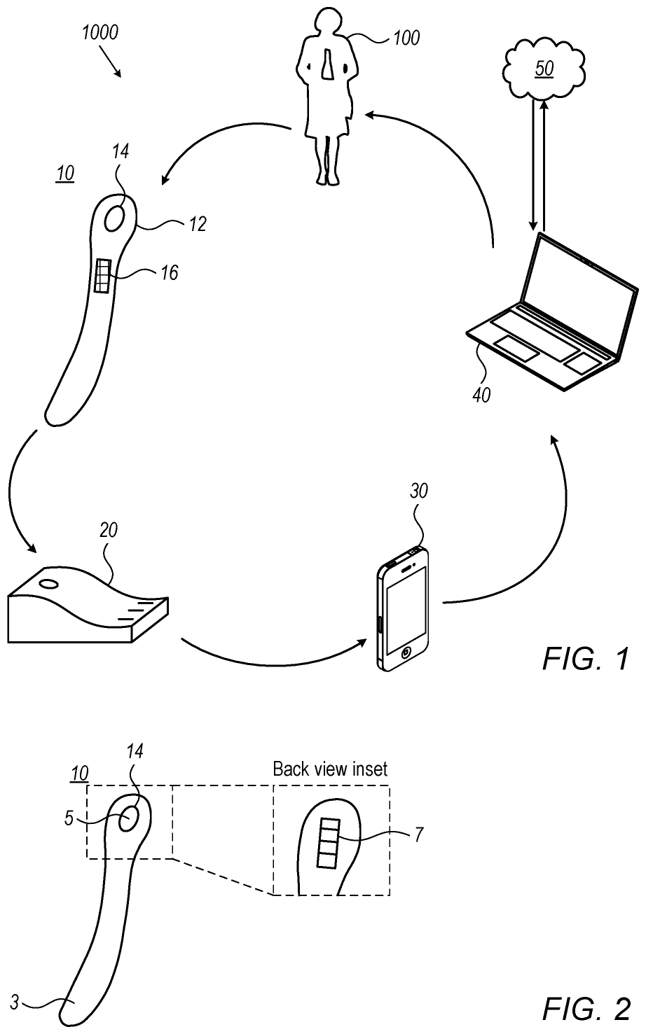

FIG. 1 is a schematic illustration of a system 1000 for diagnosis of vaginal disorders, according to an embodiment of the disclosure. According to some embodiments of the disclosure, the system 1000 comprises a sensing device 20, a collection apparatus 10, a visual data acquisition device 30 and a computer system 40. According to some embodiments of the disclosure, certain aspects of the system 1000 may be combined into one device. In an exemplary embodiment, the sensing device 20 may be integrated with the collection apparatus 10, and the visual data acquisition device 30 and the computer system 40 may be combined into a single device.

In some embodiments, there is provided an apparatus for diagnosis of vaginal disorders. In some embodiments, said apparatus comprises a collection apparatus for collecting and sampling vaginal discharge or fluid, and a sensing device for processing the sampling vaginal discharge or fluid, said sensing device comprising at least two lateral flow assays (LFAs). In some embodiments, the apparatus further includes a visual data acquisition device for capturing images of a vulva, sampling vaginal discharge or fluid and results generated by the at least two LFAs.

According to some embodiments, the collection apparatus 10 is an apparatus designed for collecting and sampling vaginal discharge or fluid. In some embodiments, the collection apparatus 10 is substantially made of polymeric material, such as polystyrene. In some embodiments, the collection apparatus 10 may be made of other suitable materials having similar physicochemical properties as polystyrene. In some embodiments, the collection apparatus may be shaped to be handheld by a user 100 and to comfortably fit the vagina of the user 100. In some embodiments, the collection apparatus 10 may comprise a vaginal end 12 with at least one swab head 14 and at least one pH sensor 16. In some embodiments, the at least one swab head 14 and the at least one pH sensor 16 comprising a collecting system. In some embodiments, the collecting system is preferably at a location that comes in contact with vaginal fluids during the collection process. According to some embodiments, the at least one swab may be a foam swab. In some embodiments, the foam swab may be a commercially available foam swab. In some embodiments, the collection apparatus 10 may comprise one, two, or three swab heads 14 and one, two or three pH sensors 16. In some embodiments, the collection apparatus 10 may comprise one swab head 14 and two pH sensors 16. In some embodiments, the collection apparatus 10 may comprise two swab heads 14 and one pH sensor 16.

According to some embodiments of the disclosure, the sensing device 20 is an apparatus for processing or analyzing vulvovaginal samples comprising at least two or at least three lateral flow assays (LFA's). In some embodiments, the sensing device 20 may comprise three, four or five LFAs. In some embodiments, the sensing device 20 may comprise at least one Candida species antigenic LFA such as for example, Candida rapid test (for example from Hangzhou ALLTEST Biotech Co. Hangzhou, China). In some embodiments, the sensing devices 20 may comprise at least one Trichomonas Vaginalis antigenic LFA, such as for example, Trichomonas rapid test (from Hangzhou ALLTEST Biotech Co. Hangzhou, China). In some embodiments, the sensing devices 20 may comprise at least one Sialidase enzyme activity LFA, such as for example, BCIN (bromo-4-chloro-3-indolyl-α-DN-acetylneuraminic acid) laced litmus paper (from MERCK).

In some embodiments of the disclosure, the sensing device 20 may also include a pH sensor, such as for example, pH indicator strip (from MERCK, Germany), or an electronic pH meter, such as for example, Mini Digital pH Meter (from General® Tools, NY USA).

In some embodiments of the disclosure, the sensing device 20 may comprise additional sensing tests kits or testing devices. The additional sensing tests kits or devices may include other antigenic tests such as for example, Chlamydia trachomatis like, Chlamydia test (from Home health UK, UK); rapid Nucleic acid amplification tests for detection of microorganism presence, such as for example, Helicase dependent DNA amplification system (from Quidel Corporation, California, USA); electronic nose sensors for detection of Vaginal amines and other molecules of vaginitis (such as MiCS-5524 from SGX sensortech), and a portable spectrometer such as Progeny handheld spectrometer (from Rigaku, Tokyo, Japan).

In some embodiments, the sensing device 20 may comprise one Candida species antigenic LFA, one Trichomonas Vaginalis antigenic LFA and one Sialidase enzyme activity LFA. In some embodiments, the sensing device 20 may comprise at least two LFAs selected from the group consisting of tests or kits for Candida species antigenic, Trichomonas vaginalis antigenic, Sialidase enzyme activity, Chlamydia trachomatis antigenic, rapid nucleic acid detection of microorganism presence, and detection of vaginal amines molecules typical of vaginitis. In some embodiments, as previously stated, the sensing device 20 may further comprise a portable spectrometer.

In accordance with some embodiments of the disclosure, a visual data acquisition device 30 is provided for capturing images of vulva, discharge and vulvovaginal fluid. In some embodiments, the visual data acquisition device 30 comprises a camera, such as for example, a mobile phone camera. In some embodiments, the visual data acquisition device 30 may comprise other sensing devices such as a gas sensor (not shown). The gas sensor may be an electronic nose or odor meter capturing gas for analysis. In some embodiments, the gas sensor may be an amine sensor or a chemiresistive sensor. The gas sensor may be based on polymer/carboxylic-functionalized single-walled carbon nanotubes. Such sensing device may further comprise an internal air pump to deliver gas to the sensing device 20 (not shown). In some embodiments, the visual data acquisition 30 comprises both a camera and the gas sensor. In some embodiments, the data captured by the visual data acquisition device 30 is transferred to computer system 40 for further analysis.

Optionally, the sensing device 20 may further comprise a central processing unit (CPU), a transient and/or fixed memory storage device, a communication device enabling the sending and/or receiving of data, and a power source such as for example, a battery or suitable power connection to visual data acquisition device 30 (such as through a USB cable) or computer system 40 or connection to mains. According to some embodiments, the sensing device 20 may communicate directly or through telecommunication networks 50 with the computer system 40 such as Personal Computer (PC) or a handheld mobile device executing a computer program application.

According to some embodiments of the disclosure, the computer system 40 is connected through a network communication device directly or indirectly to the collecting device 10, the sensing device 20 and to the visual data acquisition device 30. In some embodiments, the computer system 40 comprises a CPU, a memory storage device and a communication device. In some embodiments, the computer system 40 is programmed to receive data from one or more of the devices in system 1000 such as collecting device 10, sensing device 20 and visual data acquisition 30. In some embodiments, the computer system 40 executes a computer program stored in the storage device using the CPU to determine the probability of vaginal condition based received data. In some embodiments, the data may be presented to user 100 or to a physician (not shown) through network 50.

FIG. 2 is a schematic illustration of the collection apparatus 10 in the form of a collecting spoon, according to an embodiment of the disclosure. It is to be understood that the form or shape of the collection apparatus is not limited to the collecting spoon above. Hence, other suitable forms or shapes may be used. According to some embodiments of the disclosure, the collection apparatus 10 comprises a collecting spoon that is advantageously a specialized form for convenient sample collection and for ensuring sufficient sample quantity is collected by the user 100. In such an embodiment, the collection apparatus 10 may comprise a single swab. The single swab may be made of cotton or similar material for efficient vulvovaginal discharge collection. A pH strip 7 can also be fitted in close proximity to swab heads. The collection apparatus 10 may be made of silicone and plastic for smoothness and rigidity such that it will not be intrusive but remain firm during the collection process. In some embodiments, the collection apparatus 10 comprises a handle 3 for easy handling and a collection cup 5. In some embodiments, the collection cup 5 is a spoon like collection cup. The collection cup 5 comprises a collecting well having an opening such that a photograph of the collected specimen can be easily achieved by the user 100. In some embodiments, the collection apparatus 10 has a backside seen in inset of FIG. 2, having a pH sensor 7 (for example pH strip) mounted such that when a sample is collected, the pH strip is partly or fully immersed in the sample (for example vaginal fluid). In some embodiments, the pH strip 7 can be configured such that its sensing part is exposed into spoon inner side. In such embodiments, when sample is collected, the pH strip 7 may be exposed to vaginal fluids collected in spoon. In some embodiments, the pH readings are shown on the pH strip 7 backside that is facing back side of the collection apparatus 10. In some embodiments, the pH strip 7 backside may be made of substantially transparent material such as silicone, plastic, and the like.

pH reading is performed by reading the pH strip 7 located on the back of the collecting system 10 seen best in inset of FIG. 2. In some embodiments, an electronic pH meter may be placed in the sensing device 20, instead of the pH strip 7 and some vaginal fluid can be used as sample.

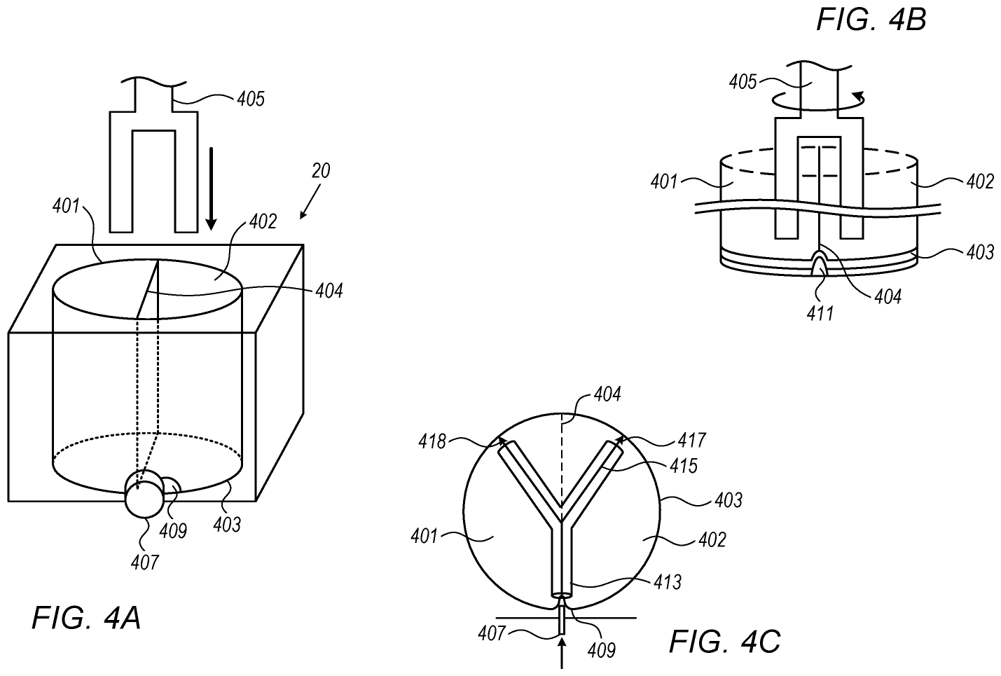

In some embodiments, the collection apparatus 10 may include a dual or a triple swab head. It is to be appreciated that the collection apparatus 10 having dual swab head would mean the collection apparatus 10 has two swab heads. Accordingly, the collection apparatus 10 having triple swab head would mean the collection apparatus 10 has three swab heads. Similar to the single swab head, the dual or triple swab head can be fabricated from cotton or similar material for efficient vulvovaginal discharge collection. A pH strip 7 can also be fitted in close proximity to swab heads. The swab heads are designed to fit sampling and mixing chambers of sensing device further discussed in FIG. 4.

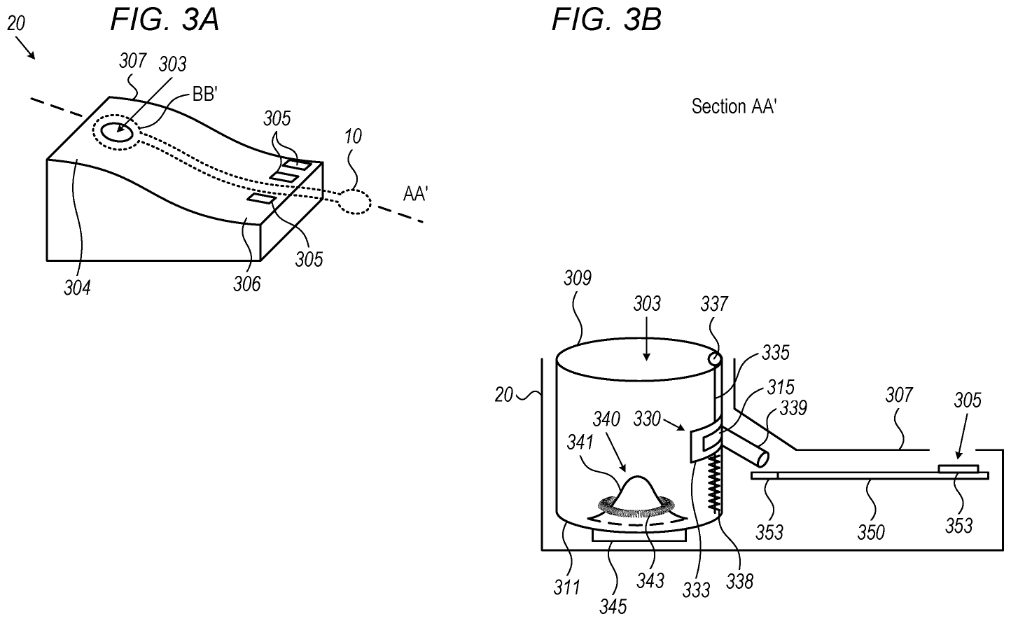

FIG. 3A is a perspective view of the sensing device 20 shown in FIG. 1 and FIG. 3B is a cross sectional view AA′ of the sensing device 20 of FIG. 3A, according to an embodiment of the disclosure. In some embodiments of the disclosure, the sensing device 20 comprises a housing 307 having a top part 304 and a flat base 306. In some embodiments, the top part 304 is elevated compared with the flat base 306. In some embodiments, the shape of the housing 307 may be designed to comport to the shape of the collection apparatus 10. In some embodiments, the shape of the housing 307 may be shaped for easy sample delivery and stability, allowing for vaginal fluids to flow from the top part 304 to the flat base 306. In other embodiments, where the collection apparatus 10 is for example a syringe or pipette, the shape of the top part 304 may be designed for use with such collection devices. In some embodiments, the sensing device 20 further comprises a sampling and mixing chamber 303 located at proximal end of the sensing device 20. In some embodiments, three or more result view ports 305 (for example three, four, five or six result view ports) are located at the distal end of the sensing device 20. A dashed line BB′ illustrates the location where the collection apparatus 10 can be placed upon and be integrated with the sensing device 20. When the collection apparatus 10 is placed upon the sensing device 20, the user 100 may place the collection apparatus 10 such that the collection cup 5 opening faces the sampling and mixing chamber 303 allowing for the collected sample to flow into sampling and mixing chamber 303. In other embodiments, the user may discharge the sample from the collection apparatus 10 into the mixing chamber 303 through the release of the sample from the syringe or pipette.

In accordance with some embodiments of the disclosure, the housing 307 shown in FIG. 3B that substantially covers inner parts of the sensing device 20, comprising sampling and mixing chamber 303, delivery conduit 339 and at least one LFA 350 (for example one, two or three LFAs). In some embodiments, the sampling and mixing chamber 303 is formed of walls 309 and bottom 311. The sampling and mixing chamber 303 may be a reservoir for holding fluid. In some embodiments, the reservoir may be a small reservoir. In some embodiments, the fluid may comprise a buffer solution, reagents and the like for preparation of the collected sample prior to delivery to the at least one LFA 350 for analysis. It is to be understood that the sampling and mixing chamber 303 may be used for mixing the sample and the reagents (or the buffer solution) prior to analyzing the sample. In some embodiments, the sampling and mixing chamber 303 is a closed chamber with a delivery aperture 315, a delivery mechanism 330, and a mixing mechanism 340 mounted at its bottom 311. In some embodiments, the sampling and mixing chamber 303 comprises an opening that corresponds to the collection cup 5, such that the collected sample flows from the collection cup 5 to the sampling and mixing chamber 303. In some embodiments, other suitable type of mixing mechanism 340 may also be used.

In some embodiments of the disclosure, the mixing mechanism 340 comprises a rod or mount like protrusion 341 of the bottom of the sampling and mixing chamber 303. In some embodiments, the mixing mechanism further comprises a round metallic wired mixer element 343 riding on the mount like protrusion 341. The mixer element 343 is rotated by force induction of simple electric motor 345 in and below the rod, powered by a power source (not shown). In some embodiments, a magnetic mechanism (not shown) can be constructed to rotate the mixer element 343. In some embodiments, the magnetic mechanism may be a manual magnetic mechanism. In some embodiments, a manual handle (not shown) may be provided at the bottom of the housing 307 connected to the mixer element 343 thus allowing the user 100 to manually rotate the mixer element 343.

In some embodiments of the disclosure, the delivery mechanism 330 includes a delivery aperture 315 from the sampling and mixing chamber 303 located on wall 309 and is covered by aperture door 333 that covers the delivery aperture 315 to advantageously prevent untimely fluid delivery to the at least one LFA 350. In some embodiments, the delivery aperture 315 is connected to a delivery conduit 339 that delivers mixed sample fluid to the at least one LFA 350. Hence, in some embodiments, the delivery conduit connects the sampling and mixing chamber 303 and the and at least one LFA 350. The at least one LFA 350 comprises a sampling pad 353 to which sample fluid is delivered. In some embodiments, the delivery conduit 339 can diverge to multiple tubes for multiple LFA's 350 as needed (as mentioned above two, three, four or five LFAs and correspondingly two, three, four or five tubes). In some embodiments, the delivery conduit 339 may be fabricated in diameter to provide sufficient amount of mixed sample to the sampling pad 353 or to multiple sampling pads 353. In some embodiments, the aperture door 333 is configured to move via a spring and rod mechanism. The aperture door 333 may be strung on a rod 335. The rod 335 may be fixed to the bottom 311 and attached loosely to the walls 309 or passed in a specialized constructed groove in said wall 309 (not shown) all in close approximation to delivery aperture 315. At the top end of the rod 335, a rounded push element 337 may be fitted. Below the aperture door 333, a spring 338 may be fitted around the rod 335. In resting phase, the spring extends from bottom part of the aperture door 333 to bottom 311.

In some embodiments of the disclosure, the vaginal fluid sample is obtained by the user 100 using collection apparatus 10. The sample is subsequently brought into sampling and mixing chamber 303. The sample is then mixed with fluids in the sampling and mixing chamber 303 by the mixing mechanism 340. When the mixing mechanism 340 operation is substantially completed, a few drops of fluid is delivered to the at least one LFA 350 through the action of delivery mechanism 330. This is achieved by pressing a push element 337, which forces the aperture door 333 to slide down on rod 335 against spring 338 force. Sliding of aperture door 333 reveals delivery aperture 315 and allows mixed sample in the sampling and mixing chamber 303 to flow through delivery tube 339 to the sampling pad 353 of the at least one LFA 350. When the mixed sample is in contact with the at least one LFA 350, the LFA process is initiated, resulting in color lines being visible on the sampling pad 353 that can be visualized or detected through view ports 305. In some embodiments, the aperture door 333 is shut via reverse action of delivery mechanism 330 via spring 338 recoil action.

In some embodiments, the sampling and mixing chamber may be at least two sampling and mixing chambers. In some embodiments, the sampling and mixing chamber may be two sampling and mixing chambers as can be seen in FIGS. 4A-4C. In such an embodiment, as seen in FIG. 4A depicting a perspective view of the two sampling and mixing chambers of the sensing device 20, two joint half-circled sampling and mixing chambers 401 and 402 are based on a hinged swivel base 403 and separated by a divider wall 404. As can be appreciated, this divider wall 404 will disallow the mixing of the liquid in chambers 401 and 402. The base 403 can freely rotate in 360 degrees of freedom and is stopped by a push rod button element 407 that is inserted into a lock recess 409 in the base 403. As can be seen from FIG. 4B shown a lateral view of the two sampling and mixing chambers of the sensing device 20, the base 403 is preferably mounted on a hinged 360 degrees pivot element 411. A top view of the two sample and mixing chambers 401 and 402 is seen in FIG. 4C. Base plate 403 is divided into two halves of sample and mixing chambers 401 and 402 by the divider wall 404. Stemming from the lock recess 409 is a Y shaped channel 413 both Y ends at and with close proximity to wall of chambers 401 and 402. Channel 413 lies at the bottom of chambers 401 and 402, divider 404 and continues in a 45-degree angle towards chambers 401 and 402 walls. Inside said channel 413 runs a push rod 415 at both its end are puncture elements 417 and 418, respectively. Other mechanism such as door and spring mechanism similar to that shown in FIG. 3 may also be used. It could be appreciated that sampling and mixing chamber may be divided to three separate chambers with adjusted rod and puncture elements to fit a multitude chamber setup. The collecting apparatus 405 is adapted to this sampling and mixing chamber consisting of two sampling heads in this example. In a three-chambers set up, a three swab head sampling collecting apparatus is envisioned. It is to be understood that in the three chambers set up, there may be two divider walls.

In operation, the collecting apparatus 405 is brought into mixing chambers 401 and 402 in such a fashion that each swab head fits into one chamber. The configuration of multiple chambers such as one illustrated in FIG. 4A advantageously allows different buffer solution or reagents to be used separately in two different chambers. User rotates the chambers 401 and 402 using a rotational force on the handle of the collection apparatus 405. Hence, advantageously, the collection apparatus 405 may have a dual function as the collecting sample apparatus and the stirrer or mixer. When mixing is done, the user pushes the push rod button 407, which locks the base 403 in the sample delivery location. Further push of the rod button and element 407 pushes the push rod 415. This in turn will push the puncture elements 417 and 418 through walls of sampling and mixing chambers 401 and 402 forming apertures thus allowing fluid sample to exit into conducting ducts leading to LFA sampling pads (both not shown) similar as previously described.

In some embodiments of the disclosure, the user 100 having vulvovaginal symptoms may interact with the computer system 40 to provide information regarding medical condition. In some embodiments, the user 100 may use the collection apparatus 10 to collect vaginal fluid sample. The user 100 may photograph the sample located in collecting device 10 using the visual data acquisition device 30. The user 100 may place a sample of collected vaginal fluids in the sensing device 20 directly or through placing the collection apparatus 10 on the sensing device 20. As described above, the sensing device 20 shows results provided by at least one LFA 350. In some embodiments, where sample is analyzed for pH, presence of organisms Candida species, Trichomonas vaginalis as well as Sialidase enzyme activity, the user 100 photographs the at least one LFA 350 showing color results and/or pH strip 7 and send said photograph(s) to the computer device 40. In some embodiments, the user 100 may also use the sensing device 20 to further acquire odor data, through the gas sensor. The same sensing device 20 may be equipped to acquire visual as well as odor data via separate sensors, such as a camera and a gas sensor. In some embodiments, the results may be recorded internally by the sensing device 20 and/or collection apparatus 10 and are transferred to the computer device 40. In some embodiments, the computer device 40 elements may be integrated in the sensing system 20 such that it will perform all tasks associated with the computing device 40 such as computing, storing, transmitting and the like. According to some embodiments, the user 100 can send photographs of the at least one LFA 350 showing color results and/or pH strip 7 to a remote health care provider (clinics, hospitals), pharmacy, and other health related organizations via remote connection.

In some embodiments, there is provided a kit comprising an apparatus as defined herein. In some embodiments, the kit is useful for diagnosing the vaginal disorders. In some embodiments, the kit comprises a collection apparatus for collecting and sampling vaginal discharge or fluid, similar to as previously described above; and a sensing device for processing the sampling vaginal discharge or fluid, wherein said sensing device comprising at least two lateral flow assays (LFAs) as previously described. In some embodiments, the kit or the apparatus described herein may be self-administered. Advantageously, the kit or the apparatus may be used in the absence of a practitioner (for example medical doctor, gynecologists). In some embodiments, the kit or the apparatus described in the present disclosure may be a disposable kit or apparatus suitable for one time use and advantageously the kit or apparatus is handy and easy-to-use. The kit or the apparatus described herein may provide a quick indication whether or not a subject is suspected to have vaginal disorders. A further test or analysis may be required to confirm if the subject indeed has the vaginal disorders.

In some embodiments, there is provided a method for diagnosing the vaginal disorders using the apparatus or the kit described herein.

It should be appreciated that the above-described methods and apparatus may be varied in many ways, including omitting, or adding elements or steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

1. An apparatus for diagnosis of vaginal disorders, comprising:
a collection apparatus for collecting and sampling vaginal discharge or fluid; and
a sensing device for processing the sampling vaginal discharge or fluid, said sensing device comprising at least two lateral flow assays (LFAs).

2. The apparatus according to claim 1, further comprising a visual data acquisition device for capturing images of a vulva, sampling vaginal discharge or fluid and results generated by the at least two LFAs.

3. The apparatus according to claim 1, wherein the collection apparatus is shaped to be handheld and to comfortably fit a vagina of a user of the apparatus for diagnosis of vaginal disorders.

4. The apparatus according to claim 1, wherein the collection apparatus further comprises a vaginal end with at least one swab head and at least one pH sensor.

5. The apparatus according to claim 1, wherein the collection apparatus is shaped as a spoon, and comprises a handle and a collection cup having an opening.

6. The apparatus according to claim 1, wherein the collection apparatus further comprises a pH sensor mounted such that when a sample is collected, the pH sensor is immersed in vaginal fluid.

7. The apparatus according to claim 6, wherein the pH sensor comprises a wall made of a transparent material.

8. The apparatus according to claim 1, wherein the at least two LFAs is selected from the group consisting of tests or kits for Candida species antigenic, Trichomonas vaginalis antigenic, sialidase enzyme activity, Chlamydia trachomatis antigenic, rapid nucleic acid detection of microorganism presence, and detection of vaginal amines molecules typical of vaginitis.

9. The apparatus according to claim 1, wherein the sensing device further comprises a portable spectrometer.

10. (canceled)

11. The apparatus according to claim 1, wherein the sensing device comprises a housing having a top part and a flat base, the top part is elevated compared with the flat base, allowing for mixed vaginal fluids to flow to the at least two LFAs and wherein the housing is shaped to comport to the shape of the collection apparatus.

12. (canceled)

13. The apparatus according to claim 1, wherein the sensing device further comprises a sampling and mixing chamber formed of walls and a bottom at a proximal end and at least two view ports a distal end.

14. The apparatus according to claim 1, wherein the sensing device further comprises a delivery mechanism, the delivery mechanism comprises a delivery conduit connecting the sampling and mixing chamber and at least two LFAs.

15. The apparatus according to claim 13, wherein the sampling and mixing chamber further comprises:
a buffer solution or reagents for preparation of collected sample prior to delivery to the at least two LFAs; and
a mixing mechanism.

16. (canceled)

17. The apparatus according to claim 14, wherein the delivery mechanism comprises at least two tubes extending from the sampling and mixing chamber.

18. (canceled)

19. (canceled)

20. The apparatus according to claim 13, wherein the sampling and mixing chamber has at least two sampling and mixing chambers separated by a divider wall.

21. The apparatus according to claim 20, wherein the at least two sampling and mixing chambers are placed on a rotatable base.

22. The apparatus according to claim 20, wherein the at least two sampling and mixing chambers comprising puncture elements, wherein apertures are formed when said puncture elements are pushed through walls of the sampling and mixing chamber.

23. The apparatus according to claim 20, wherein the collection apparatus further comprises a vaginal end with at least two swab heads.

24. A kit for diagnosing vaginal disorders comprising:
a collection apparatus for collecting and sampling vaginal discharge or fluid and
a sensing device for processing the sampling vaginal discharge or fluid, wherein said sensing device comprising at least two LFAs.

25. The kit according to claim 24, wherein the at least two LFAs is selected from the group consisting of tests or kits for Candida species antigenic, Trichomonas vaginalis antigenic, sialidase enzyme activity, Chlamydia trachomatis antigenic, rapid nucleic acid detection of microorganism presence, and detection of vaginal amines molecules typical of vaginitis.
